(19) United States
(12) Patent Application Publication
(10) Pub. No.: US 2023/0248807 A1
(43) Pub. Date: Aug. 10, 2023

(54) HP-HMG FOR USE IN THE TREATMENT OF INFERTILITY IN A PATIENT WITH POLYCYSTIC OVARY SYNDROME

(71) Applicant: Ferring B.V., Hoofddorp (NL)

(72) Inventor: Patrick HEISER, Parsippany, NJ (US)

(73) Assignee: Ferring B.V., Hoofddorp (NL)

(21) Appl. No.: 18/012,753

(22) PCT Filed: Jun. 26, 2020
(86) PCT No.: PCT/US2020/039745

§ 371 Date: Dec. 23, 2022

(51) Int. Cl. A61K 38/24; A61P 15/08; C12N 5/075

(52) U.S. Cl. CPC A61K 38/24; A61P 15/08; C12N 5/0609

(57) ABSTRACT

Described are assisted reproductive technology compositions and methods using highly purified menotropin (HP-hMG) to stimulate follicle development, particularly in women who have been diagnosed with oligoovulation and/or PCOS and who are predicted to have a high ovarian response to controlled ovarian stimulation.

### FIELD OF INVENTION

The invention described herein relates to assisted reproductive technology. In particular, described herein are compositions and methods for treating infertility, including controlled ovarian stimulation methods that may be particularly useful for women who experience oligoovulation and/or who have Polycystic Ovarian Syndrome (PCOS) and are predicted to have a high ovarian response to controlled ovarian stimulation.

### BACKGROUND OF THE INVENTION

Assisted reproductive technology (ART) procedures generally involve stimulating egg development and maturation, harvesting eggs from a woman's ovaries, combining them with sperm in vitro, and transferring them to a woman's uterus (the donor or another woman). Success of ART is hampered by maternal and perinatal risks associated with the stimulation of egg development and maturation, such as ovarian hyperstimulation syndrome (OHSS) and ectopic pregnancy. Other concerns that arise in ART are the production of quality embryos and euploid blastocysts to support ongoing pregnancy rates and live birth rates.

Gonadotropins, such as menotropin (e.g., human menopausal gonadotropin, or hMG), follicle-stimulating hormone (FSH) and luteinizing hormone (LH), have been used for controlled ovarian stimulation (COS), and highly purified menotropin (HP-hMG) and recombinant human FSH (rFSH) have been used more recently. HP-hMG provides FSH and exogenous LH activity mainly in the form of human chorionic gonadotrophin (hCG). The efficacy of ovarian stimulation protocols may be enhanced using long gonadotropin hormone releasing hormone (GnRH) agonists or GnRH antagonists for cycle control. See, e.g., Devroey et al., Fertility and Sterility 97: 561-71 (2012). Ziebe et al., Human Reproduction 22(9) 2404-13 (2007), reported that the use of HP-hMG versus rFSH could impact the morphology of embryos, and observed improved implantation, ongoing pregnancy and live birth rates among the top-quality embryos (based on visual assessment) derived from stimulation with HP-hMG compared with Chinese hamster ovary cell (CHO cell)-derived rFSH (GONAL-F).

Because patient responses to ovarian stimulation vary widely, treatments often are individualized. For example, individualization may be based on predicted ovarian response to gonadotropin stimulation, which forecasts poor, normal or high response. High ovarian responders usually are defined as women who produce high numbers of developing follicles following a standard protocol of controlled ovarian stimulation (COS). Although these patients are generally considered good candidates for ART, high ovarian response may be associated with lower implantation rates and higher miscarriage rates, and thus a decreased chance of successful outcome as compared with a normal ovarian response. These high responders also are at greater risk for OHSS and the complications associated therewith.

Efforts to develop improved ART methods for predicted high responders have involved exploring milder stimulation protocols. For example, Rubio et al., Human Reproduction 25(9): 2290-97 (2010), reported that decreasing the gonadotropin dose administered to high responders could improve fertilization rates and embryo quality, although the lower doses resulted in fewer oocytes. Other efforts have considered whether the specific gonadotropin used impacts the results. For example, Arce et al., Gyn. Endocrin. 30(6): 444-50 (2014), reported that among predicted high responders (subjects having an AMH≥5.2 ng/ml) the group stimulated with CHO cell-derived rFSH (GONAL-F) had significantly more oocytes retrieved, but a significantly lower live birth rate per cycle as compared to the group stimulated with HP-hMG (20% vs. 33% in the MERIT “long agonist” clinical trial; 23% vs. 34% in the MEGASET “antagonist” trial); see also La Marca et al., Fertility and Sterility 0-169 (2012) (same).

A woman with an average menstrual cycle typically ovulates or releases a mature egg once a month, about halfway through her cycle. Oligoovulation refers to when ovulation occurs infrequently or irregularly, and usually is classified as having eight (8) or fewer menstrual cycles (periods) in a year. Oligoovulation is one of the most common causes of infertility for women.

There is a need for improved assisted reproductive technology methods, particularly for women who experience oligoovulation and/or who have Polycystic Ovarian Syndrome (PCOS) and who are predicted to have a high ovarian response to controlled ovarian stimulation.

### SUMMARY OF THE INVENTION

The compositions and methods described herein stem from the surprising and unexpected discovery that patients who experience oligoovulation, including women who experience oligoovulation due to PCOS, and who are predicted to have a high ovarian response to controlled ovarian stimulation (e.g., predicted to be high-responders) and undergo infertility treatment with a controlled ovarian stimulation protocol that uses hMG as the gonadotropin have a significantly higher ongoing pregnancy rate compared to those who undergo infertility treatment with a controlled ovarian stimulation protocol that uses rFSH as the gonadotropin. In other words, the compositions and methods described herein stem from the surprising and unexpected discovery that selection of patients diagnosed with oligoovulation (including women diagnosed with oligoovulation and PCOS) who are predicted high responders (including patients who have elevated baseline levels of AMH, estradiol, LH, and/or testosterone as disclosed herein) for controlled ovarian stimulation infertility treatment with HP-hMG, rather than rFSH, as the gonadotropin may be associated with higher ongoing pregnancy rate.

Provided herein are compositions comprising highly purified menotropin (HP-hMG) for use in the treatment of infertility, optionally by controlled ovarian stimulation, in a patient with polycystic ovary syndrome (PCOS), wherein the patient has a serum anti-Müllerian hormone (AMH) level≥35.7±0.5 pmol/L (≥5.0±0.2 ng/ml) prior to treatment/stimulation.

Also provided herein are compositions comprising highly purified menotropin (HP-hMG) for use in the treatment of infertility, optionally by controlled ovarian stimulation, in a patient with oligoovulation caused by polycystic ovary syndrome (PCOS), wherein the patient has a serum anti-Müllerian hormone (AMH) level≥35.7±0.5 pmol/L (≥5.0±0.2 ng/ml) prior to treatment/stimulation.

In any embodiments, the composition for use may comprise 75 to 450 IU HP-hMG. In any embodiments, the treatment of infertility may comprise administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

In any embodiments, the treatment of infertility may comprise identifying (e.g., diagnosing) a patient who has (a) a serum anti-Müllerian hormone (AMH) level≥35.7±0.5 pmol/L (≥5.0±0.2 ng/ml) prior to treatment/stimulation and (b) a serum estradiol level of ≥145 pmol/L (e.g., a serum estradiol level of ≥150 pmol/L) prior to treatment/stimulation, and optionally also has one or both of (c) a serum testosterone level of ≥1.10 nmol/L (e.g., a serum testosterone level of ≥1.14 nmol/L) prior to treatment/stimulation, and (d) a serum luteinizing hormone (LH) level of ≥7 U/L (e.g., a serum luteinizing hormone of ≥7.55 U/L) prior to treatment/stimulation; and administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

Also provided are compositions comprising highly purified menotropin (HP-hMG) for use in the treatment of infertility, optionally by controlled ovarian stimulation, in a patient with polycystic ovary syndrome (PCOS) and a serum anti-Müllerian hormone (AMH) level≥35.7±0.5 pmol/L (≥5.0±0.2 ng/ml) prior to treatment/stimulation, the treatment comprising identifying (e.g., diagnosing) a patient with PCOS who has a serum anti-Müllerian hormone (AMH) level≥35.7±0.5 pmol/L (≥5.0±0.2 ng/ml) prior to treatment/stimulation; and administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

Also provided are compositions comprising highly purified menotropin (HP-hMG) for use in the treatment of infertility, optionally by controlled ovarian stimulation, in a patient with oligoovulation caused by polycystic ovary syndrome (PCOS) and a serum anti-Müllerian hormone (AMH) level≥35.7±0.5 pmol/L (≥5.0±0.2 ng/ml) prior to treatment/stimulation, the treatment comprising identifying (e.g., diagnosing) a patient with oligoovulation caused by PCOS who has a serum anti-Müllerian hormone (AMH) level≥35.7±0.5 pmol/L (≥5.0±0.2 ng/ml) prior to treatment/stimulation; and administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

In any embodiments, the treatment of infertility may comprises identifying (e.g., diagnosing) a patient who has (a) a serum anti-Müllerian hormone (AMH) level≥35.7±0.5 pmol/L (≥5.0±0.2 ng/ml) prior to treatment/stimulation and (b) a serum estradiol level of ≥145 pmol/L (e.g., a serum estradiol level of ≥150 pmol/L) prior to treatment/stimulation, and optionally also has one or both of (c) a serum testosterone level of ≥1.10 nmol/L (e.g., a serum testosterone level of ≥1.14 nmol/L) prior to treatment/stimulation, and (d) a serum luteinizing hormone (LH) level of ≥7 U/L (e.g., a serum luteinizing hormone of ≥7.55 U/L) prior to treatment/stimulation; and administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

With respect to the compositions for use as disclosed herein, the treatment of infertility as disclosed herein increases ongoing pregnancy rate compared to treatment with recombinant follicle-stimulating hormone (rFSH).

In any embodiments of the compositions for use disclosed herein, the treatment may further comprise triggering final follicular maturation by administering hCG or a GnRH agonist, optionally supplemented with hCG.

In any embodiment of the compositions for use disclosed herein, the treatment may be a fresh transfer method further comprising retrieving oocyte(s), fertilizing the oocyte(s), allowing the fertilized oocyte(s) to develop to the blastocyst stage, optionally assessing the quality/morphology of the blastocyst(s), and implanting a fresh blastocyst (optionally selected based on, e.g., visual assessment of quality/morphology) in a uterus.

In any embodiment of the compositions for use disclosed herein, the treatment may be a frozen transfer method further comprising retrieving oocyte(s), fertilizing the oocyte(s), allowing the fertilized oocyte(s) to develop to the blastocyst stage, optionally assessing the chromosomal quality of the blastocyst(s), freezing one or more or all blastocyst(s), and implanting a thawed frozen blastocyst (e.g., a euploid blastocyst selected based on chromosomal assessment) in a uterus.

In any embodiment of the compositions for use disclosed herein, the treatment may further comprise retrieving oocyte(s), freezing unfertilized oocytes(s), subsequently thawing one or more oocyte(s), fertilizing one or more or all thawed oocyte(s), allowing fertilized oocyte(s) to develop to the blastocyst stage, optionally assessing the quality/morphology of the blastocyst(s), and implanting a blastocyst (optionally selected based on, e.g., visual assessment of quality/morphology) in a uterus; or retrieving oocyte(s), freezing unfertilized oocytes(s), subsequently thawing one or more frozen oocytes, fertilizing one or more or all thawed oocyte(s), allowing fertilized oocyte(s) to develop to the blastocyst stage, optionally assessing chromosomal quality of the blastocyst(s), freezing one or more or all blastocyst(s), and implanting a thawed-frozen blastocyst (e.g., a euploid blastocyst selected based on chromosomal assessment) in a uterus.

In any embodiment of the compositions for use disclosed herein, the treatment may further comprises a step of administering a GnRH antagonist starting on day 6 of treatment.

In any embodiment of the compositions for use disclosed herein, the patient is not anovulatory, is 21-35 years old, and has a BMI of 18-30 kg/m2 at the start of treatment.

Also provided are assisted reproductive technology methods for treating a woman diagnosed with one or both of oligoovulation and PCOS and predicted to have a high ovarian response to controlled ovarian stimulation, comprising identifying a woman as diagnosed with one or both of oligoovulation and PCOS and as having a serum anti-Müllerian hormone (AMH) level 35.7±0.5 pmol/L (≥5.0±0.2 ng/ml), and conducting controlled ovarian stimulation by administering to the identified woman an amount of highly purified menotropin (HP-hMG) effective to stimulate follicle development. In some embodiments, the woman is identified as being diagnosed with oligoovulation. In some embodiments, the woman is identified as being diagnosed with oligoovulation caused by PCOS. In some embodiments, the woman is identified as being diagnosed with PCOS. In some embodiments, the woman is identified as being diagnosed with oligoovulation and PCOS. The methods may further comprise identifying the woman as having one or more of (i) a serum luteinizing hormone (LH) level of greater than or equal to 7 U/L prior to treatment/stimulation, (ii) a serum testosterone level of greater than or equal to 1.10 nmol/L prior to treatment/stimulation, and (iii) a serum estradiol level of greater than or equal to 145 pmol/L prior to treatment/stimulation. The methods are effective to increase ongoing pregnancy rate after in vitro fertilization compared to treatment/controlled ovarian stimulation by administration of recombinant follicle-stimulating hormone (rFSH).

The HP-hMG may be administered at a dose of 75 to 450 IU hMG per day. The HP-hMG may be administered at a dose of 150 IU hMG per day, e.g., from day 1 to at least day 5 of treatment.

The methods may further comprise administering a gonadotropin-releasing hormone antagonist (GnRH antagonist) starting on day 6 of treatment/stimulation.

The methods may further comprise triggering final follicular maturation by administering human chorionic gonadotropin (hCG) or a gonadotropin-releasing hormone agonist (GnRH agonist), optionally supplemented with hCG.

The methods may further comprise one of (a) retrieving oocyte(s), fertilizing the oocyte(s), allowing the fertilized oocyte(s) to develop to the blastocyst stage, optionally assessing the quality/morphology of the blastocyst(s), and implanting a fresh blastocyst (optionally selected based on, e.g., visual assessment of quality/morphology) in a uterus; or (b) retrieving oocyte(s), fertilizing the oocyte(s), allowing the fertilized oocyte(s) to develop to the blastocyst stage, optionally assessing chromosomal quality of the blastocyst(s), freezing one or more or all blastocyst(s), and implanting a thawed-frozen blastocyst (e.g., a euploid blastocyst selected based on chromosomal assessment) in a uterus; or (c) retrieving oocyte(s), freezing unfertilized oocytes(s), subsequently thawing one or more oocyte(s), fertilizing one or more or all thawed oocyte(s), allowing fertilized oocyte(s) to develop to the blastocyst stage, optionally assessing the quality/morphology of the blastocyst(s), and implanting a blastocyst (optionally selected based on, e.g., visual assessment of quality/morphology) in a uterus; or (d) retrieving oocyte(s), freezing unfertilized oocytes(s), subsequently thawing one or more frozen oocytes, fertilizing one or more or all thawed oocyte(s), allowing fertilized oocyte(s) to develop to the blastocyst stage, optionally assessing chromosomal quality of the blastocyst(s), freezing one or more or all blastocyst(s), and implanting a thawed-frozen blastocyst (e.g., a euploid blastocyst selected based on chromosomal assessment) in a uterus.

The woman may be not anovulatory, 21-35 years old, and have a BMI of 18-30 kg/m2 at the start of treatment.

Also provided herein is the use of HP-hMG in the manufacture of a medicament for the treatment of infertility in a woman identified as being diagnosed with oligoovulation and/or PCOS who has a serum AMH level≥35.7±0.5 pmol/L (≥5.0±0.2 ng/ml) prior to treatment/stimulation, wherein the treatment comprises administering to the identified woman an amount of highly purified menotropin (HP-hMG) effective to stimulate follicle development. The treatment may further comprise, prior to the administering, identifying the woman as having one or more of (i) a serum luteinizing hormone (LH) level of greater than or equal to 7 U/L prior to treatment/stimulation, (ii) a serum testosterone level of greater than or equal to 1.10 nmol/L prior to treatment/stimulation, and (iii) a serum estradiol level of greater than or equal to 145 pmol/L prior to treatment/stimulation. The treatment is effective to increase ongoing pregnancy rate after in vitro fertilization compared to treatment/stimulation by administration of recombinant follicle-stimulating hormone (rFSH).

The foregoing general description is exemplary and explanatory and intended to provide further explanation of the invention. For detailed understanding of the invention, reference is made to the following detailed description. Other objects, advantages and novel features will be readily apparent to those skilled in the art from the following detailed description.

### DETAILED DESCRIPTION

Described herein are assisted reproductive technology methods, e.g., methods for treating infertility, in patients who are diagnosed with oligoovulation and/or PCOS. In particular, described herein are controlled ovarian stimulation (COS) methods that may be particularly useful for women who are diagnosed with oligoovulation and/or PCOS (including women who experience oligoovulation due to PCOS or who are diagnosed with oligoovulation and PCOS), and who are predicted to have a high ovarian response to controlled ovarian stimulation (e.g., predicted to be high-responders), including women who have baseline levels of AMH, estradiol, LH, and/or testosterone as disclosed herein. As shown in Example 1, the methods are useful for increasing ongoing pregnancy rates.

The present invention is based on the unexpected finding by the inventor that the use of highly purified menotropin (HP-hMG) for treatment by COS of patients predicted to be high-responders who have been diagnosed with oligoovulation (including women who experience oligoovulation due to PCOS), improves ongoing pregnancy rate. As reported in Example 1 below, patients predicted to be high-responders diagnosed with oligoovulation (including women who experience oligoovulation due to PCOS) and treated with HP-hMG as the gonadotropin (N=50) had a 19.2% higher ongoing pregnancy rate (95% confidence interval 1.2%-37.3%) compared to those treated with rFSH as the gonadotropin (N=56). These patients had baseline serum levels of AMH≥35.7 pmol/L (≥5.0 ng/ml), of LH≥7 U/L, of testosterone ≥1.10 nmol/L, and baseline serum estradiol levels ≥145 pmol/L. As discussed in Example 1 below, based on, e.g., their serum AMH, estradiol, LH, and testosterone levels, it is likely this patient population included patients with PCOS, e.g., patients whose oligoovulation was due to PCOS. Based on these findings, the compositions and methods disclosed herein are based on the selection of predicted high responder patients diagnosed with oligoovulation and/or PCOS, including predicted high responder patients diagnosed with oligoovulation and/or PCOS having baseline serum levels of AMH ≥35.7±0.5 pmol/L (≥5.0±0.2 ng/ml), of estradiol ≥145 pmol/L, of LH≥7 U/L, and/or of testosterone ≥1.10 nmol/L, for treatment with HP-hMG, rather than FSH, in order to achieve higher ongoing pregnancy rate.

### Definitions

Technical and scientific terms used herein have the meanings commonly understood by one of ordinary skill in the art of assisted reproductive technology to which the present invention pertains, unless otherwise defined. Reference is made herein to various methodologies known to those of ordinary skill in the art. Unless otherwise specified, any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

As used herein, the term “about” means that the number or range is not limited to the exact number or range set forth, but encompass ranges around the recited number or range as will be understood by persons of ordinary skill in the art depending on the context in which the number or range is used. Unless otherwise apparent from the context or convention in the art, “about” mean up to plus or minus 10% of the particular term.

As used herein, the term “oligoovulation” refers to infrequent or irregular ovulation amounting to eight (8) or fewer menstrual cycles (periods) per year, including women with cycles of ≥31 days. As used herein, the phrases a patient “identified with oligoovulation” or “diagnosed with oligoovulation” and a patient “with oligoovulation,” are used interchangeably to refer to a patient who has 8 or fewer menstrual cycles (periods) in a year, and excludes anovulatory patients. Oligoovulation is one of the most common causes of infertility for women.

As used herein, the term “anovulatory” or “anovulation” refers to a patient whose ovaries do not release an oocyte during a menstrual cycle. Therefore, ovulation does not take place. Chronic anovulation is a common cause of infertility. In general, the patient for the compositions and methods described herein is not an anovulatory patient.

As used here “Polycystic Ovarian Syndrome” or “PCOS” refers to a hormonal disorder characterized by two or more of elevated levels of testosterone, polycystic ovaries, and ovulatory dysfunction (such as infrequent, irregular and/or prolonged menstrual cycles). PCOS may be diagnosed according to the Rotterdam criteria, based on the presence of at least two of (i) hyperandrogenism, (ii) ovulatory dysfunction, and (iii) polycystic ovaries, with other causes of hyperandrogenism or ovulatory dysfunction ruled out.

As used herein “ongoing pregnancy” refers to pregnancy with a viable fetus and detectable fetal heartbeat at 10-11 weeks gestation, e.g., at 8-9 weeks post blastocyst/embryo transfer.

As used herein “clinical pregnancy” refers to gestation and a detectable fetal heartbeat at 5-6 weeks gestation, e.g., at 3-4 weeks post blastocyst/embryo transfer.

As used herein, “woman” refers to an adult female human. Typically, a woman treated in accordance with the compositions and methods described herein is 35 years old or younger, has a serum level of anti-Müllerian hormone (AMH)≥35.7±0.5 pmol/L (≥5.0±0.2 ng/ml) when measured using a Beckmann-Coulter Gen 2 assay as described in Arce et al., Fertility and Sterility 99: 1644-53 (2013), or an equivalent AMH level assessed by a different method, and a BMI of 30 kg/m² or less. In some embodiments, a woman treated in accordance with the compositions and methods described herein is identified, prior to treatment, as being 21-35 years old. In some embodiments, a woman treated in accordance with the compositions and methods described herein is identified, prior to treatment, as being 35 years old or younger, or 34 years old or younger. In some embodiments, a woman treated in accordance with the methods described herein is identified, prior to treatment, as being 21-34 years old, or 21-33 years old, or 21-32 years old, or 21-31 years old. In some embodiments, a woman treated in accordance with the methods described herein is identified, prior to treatment, as having a BMI of 18-30 kg/m². In some embodiments, a woman treated in accordance with the methods described herein is identified, prior to treatment, as having a BMI of 38 kg/m² or less, 36 kg/m² or less, 34 kg/m² or less, 32 kg/m² or less, 30 kg/m² or less, or 28 kg/m² or less, such as BMI of 18-38, 18-36, 18-34, 18-32, 18-30, or 18-28 kg/m². In some embodiments, a woman treated in accordance with the methods described herein is identified as having a BMI of 18-25 kg/m², 18-26 kg/m², 18-27 kg/m², 18-28 kg/m², 18-29 kg/m², or 18-30 kg/m².

As used herein, subjects classified as being “predicted to have a high ovarian response to controlled ovarian stimulation” or as a “predicted high responder” refers to women who are likely to develop high numbers of follicles or oocytes following a standard protocol of controlled ovarian stimulation (COS), such as women with a greater than average likelihood of producing 15 or more oocytes. Women may be identified as being predicted high responders if they have generated 15 or more oocytes in a previous ART cycle, e.g., in a previous COS treatment. Additionally or alternatively, women may be identified as being predicted high responders if they are considered to be at risk of developing OHSS. Additionally or alternatively, women may be identified as being predicted high responders if they have a serum level of anti-Müllerian hormone (AMH)≥35.7±0.5 pmol/L (≥5.0±0.2 ng/ml), such as a serum AMH level≥35.7±0.5 pmol/L (≥5.0±0.2 ng/ml), when measured using a Beckmann-Coulter Gen 2 assay as described in Arce et al., Fertility and Sterility 99: 1644-53 (2013), or an equivalent AMH level assessed by a different method.

The term “menotropin” as used herein includes human menopausal gonadotropin or “hMG,” including “highly purified menotropin” or “HP-hMG.” As used herein, the terms “highly purified menotropin” and “HP-hMG” refer to a highly purified hMG product that includes both follicle stimulating hormone (FSH) and human chorionic gonadotropin (hCG)-driven luteinizing hormone (LH) activity, including hMG products wherein most of the LH activity is provided by hCG, including products wherein ≥90%, or ≥95%, of the LH activity is provided by hCG. See, e.g., Foutouh et al., Reproductive BioMed. Online, 14(2): 145-47 (2007); Wolfenson et al., Reprod. BioMed. Online, 10(4): 442-54 (2005). In some embodiments, the HP-hMG is the HP-hMG product available under the trademark MENOPUR® from Ferring Pharmaceuticals, Inc., which contains FSH and hCG-driven LH activity, wherein ≥95%, of the LH activity is provided by hCG (pituitary hCG), as assessed by immunoreactivity. See, e.g., Arce and Smitz, Human Fertility, 14(3): 192-99 (2011). As reconstituted for use, one vial of MENOPUR® (75 IU HP-hMG) contains 75 IU FSH activity and 75 IU LH activity, wherein hCG contributes about 70 IU of the LH activity.

The term “GnRH agonist” as used herein includes gonadotropin-releasing hormone (GnRH) agonists such as buserelin (e.g., SUPRECUR®), leuprorelin (e.g., leuprolide acetate, e.g., LUPRON®), nafarelin (e.g., SYNAREL®), and triptorelin (e.g., TRELSTAR®).

The term “GnRH antagonist” as used herein includes gonadotropin-releasing hormone (GnRH) antagonists, such as ganirelix acetate (e.g., ORGALUTRAN®) and cetrorelix acetate (e.g., CETROTIDE®), which block the action of GnRH by competitive blocking of the GnRH receptors on pituitary gonadotropes, and thus prevent gonadotropin production/release and premature ovulation (release of eggs).

As used herein, the phrase “effective amount” refers to a dosage determined to provide the specific pharmacological effect for which the drug is administered in a subject in need of such treatment. It is emphasized that a therapeutically effective amount will not always be effective in treating the conditions described herein in a given patient, even though such dosage is deemed to be a therapeutically effective amount by those of skill in the art. For convenience only, exemplary dosages and therapeutically effective amounts are provided below with reference to adult female human subjects. Those skilled in the art can adjust such amounts in accordance with standard practices as needed to treat a specific subject and/or condition/disease.

Assisted Reproductive Technology Methods

The treatment methods described herein are useful in any reproductive technology methods that involve controlled ovarian stimulation (COS), such as for in vitro fertilization, including in vitro fertilization by intra-cytoplasmic sperm injection (ICSI), methods involving fresh transfer of fertilized eggs (e.g., blastocysts/embryos), methods involving freezing fertilized eggs for later implantation, and methods involving freezing unfertilized oocytes for later fertilization.

As noted above, the present invention provides reproductive technology compositions and methods that involve using highly purified menotropin (HP-hMG) as the gonadotropin for COS in women with oligoovulation and/or PCOS (including women who experience oligoovulation due to PCOS and women diagnosed with oligoovulation and PCOS) who are predicted to have a high ovarian response to COS and are undergoing COS. As also noted above, for the purposes of the compositions and methods disclosed herein, women may be identified as being predicted to have a high ovarian response to COS based on having a high ovarian response in a previous ART cycle, e.g., in a previous COS treatment, or if they have a serum level of anti-Müllerian hormone (AMH) ≥35.7±0.5 pmol/L (≥5.0±0.2 ng/ml), such as a serum AMH level≥35.7±0.5 pmol/L (≥5.0±0.2 ng/ml) when measured using a Beckmann-Coulter Gen 2 assay as described in Arce et al., Fertility and Sterility 99: 1644-53 (2013), or an equivalent AMH level assessed by a different method. Serum levels of AMH are a surrogate marker for functional ovarian follicle reserve, and a positive correlation between serum levels of AMH and ovarian response (e.g., oocyte yield) have been reported. Id. In accordance with the compositions and methods described herein, women typically are identified as being predicted high responders based on serum AMH level.

It will be appreciated that, in any example of the invention or in any embodiment of the compositions and methods disclosed herein, a step of identifying (e.g., diagnosing) a patient who has a serum anti-Müllerian hormone (AMH) level≥35.7±0.5 pmol/L (≥5.0±0.2 ng/ml) prior to treatment/stimulation may be substituted for, or augmented by, a step of identifying, prior to treatment/stimulation, a patient as having generated 15 or more oocytes in a previous ART cycle, e.g., in a previous COS treatment, or identifying, prior to treatment/stimulation, a patient considered to be at risk of developing OHSS.

An assisted reproductive technology method as described herein comprises conducting controlled ovarian stimulation in a woman diagnosed with oligoovulation and/or PCOS (including women who experience oligoovulation due to PCOS and women diagnosed with oligoovulation and PCOS) and predicted to have a high ovarian response to controlled ovarian stimulation, by using HP-hMG to stimulate follicle development. In any embodiments described herein, the HP-hMG may be MENOPUR®.

The treatment methods may comprise, prior to conducting controlled ovarian stimulation, identifying the woman as being diagnosed with oligoovulation and/or PCOS (including oligoovulation due to PCOS). Thus, in some embodiments, the woman is identified as being diagnosed with oligoovulation, in some embodiments the woman is identified as being diagnosed with oligoovulation caused by PCOS, in some embodiments the woman is identified as being diagnosed with PCOS, and in some embodiments the woman is identified as being diagnosed with oligoovulation and PCOS.

The treatment methods may further comprise, prior to conducting controlled ovarian stimulation, identifying the woman as being predicted to have a high ovarian response to controlled ovarian stimulation. Thus, an assisted reproductive technology method as described herein may comprise selecting a woman diagnosed with oligoovulation and/or PCOS and, prior to conducting controlled ovarian stimulation, identifying the woman as predicted to have a high ovarian response to controlled ovarian stimulation, such as by determining the woman has a serum AMH level greater than or equal to 35.7±0.5 pmol/L (≥5.0±0.2 ng/ml), when measured using a Beckmann-Coulter Gen 2 assay, or a comparable AMH level measured by a different method. In any embodiments, the woman may have, or be identified as having a serum AMH level greater than or equal to 35.7±0.5 pmol/L (≥5.0±0.2 ng/ml), when measured using a Beckmann-Coulter Gen 2 assay, or a comparable AMH level measured by a different method. In any embodiments, the patient (e.g., woman) is not an anovulatory woman.

Additionally or alternatively, in any embodiments, the woman may have, or may be identified as having a serum estradiol level ≥145 pmol/L prior to treatment/stimulation, or a serum estradiol level of ≥150 pmol/L prior to treatment/stimulation. Additionally or alternatively, in any embodiments, the woman may have, or may be identified as having, one or more of a serum luteinizing hormone (LH) level of greater than or equal to 7 U/L prior to treatment/stimulation (or a serum luteinizing hormone of ≥7.55 ti/L prior to treatment/stimulation) and a serum testosterone level of greater than or equal to 1.10 nmol/L prior to treatment/stimulation (or a serum testosterone level of ≥1.14 nmol/L prior to treatment/stimulation).

Thus, in any embodiments, the woman may have, or may be identified as having, prior to treatment/stimulation, one or more or all of (a) a serum anti-Müllerian hormone (AMH) level ≥35.7±0.5 pmol/L (≥5.0±0.2 ng/ml); (b) a serum estradiol level of ≥145 pmol/L (e.g., a serum estradiol level of ≥150 pmol/L); (c) a serum testosterone level of ≥1.10 nmol/L (e.g., a serum testosterone level of ≥1.14 nmol/L), and (d) a serum luteinizing hormone (LH) level of ≥7 U/L (e.g., a serum luteinizing hormone of ≥7.55 U/I). In some embodiments, the woman may have, or may be identified as having, prior to treatment/stimulation, (a) a serum anti-Müllerian hormone (AMH) level≥35.7±0.5 pmol/L (≥5.0±0.2 ng/ml) and (b) a serum estradiol level of ≥1.45 pmol/7, (e.g., a serum estradiol level of ≥150 pmol/L). In some embodiments, the woman may have, or may be identified as having, prior to treatment/stimulation, (a) a serum anti-Müllerian hormone (AMH) level≥35.7±0.5 pmol/L (≥5.0±0.2 ng/ml) and (b) a serum estradiol level of ≥145 pmol/L (e.g., a serum estradiol level of ≥150 pmol/L), and, optionally one or both of (c) a serum testosterone level of ≥1.10 nmol/L (e.g., a serum testosterone level of ≥1.14 nmol/L), and (d) a serum luteinizing hormone (LH) level of ≥7 U/L (e.g., a serum luteinizing hormone of ≥7.55 U/L).

The methods include administering HP-hMG to the subject in an amount effective to stimulate follicle development, for example, from about 75 IU/day to about 450 IU/day, including 75 IU/day, 150 IU/day, 225 IU/day, 300 IU/day, 375 IU/day or 450 IU/day. Typically, a starting dose of HP-hMG is 150 IU/day, but may range from 75 IU/day to 225 IU/day. The HP-hMG administration typically is commenced on day 2 or day 3 of the patient's menstrual cycle, such that treatment day 1 (also referred to herein as stimulation day 1) occurs on day 2 or day 3 of the patient's menstrual cycle. As noted above, pharmaceutical compositions comprising HP-hMG are available commercially, such as the MENOPUR® product sold by Ferring Pharmaceuticals, Inc., which is formulated for subcutaneous injection. Administration of HP-hMG continues daily until the desired level of follicle production is reached, for a total stimulation period of from about 1 to about 20 days, typically for a total stimulation period of from 8 to 12 days, more specifically typically for about 9-11 days, including for about 10 days.

It is known in the art to adjust gonadotropin dosing (e.g., increase or decrease HP-hMG or rFSH dosing) during the stimulation period based on the subject's ovarian (follicular) response, which may be assessed, for example, by transvaginal ultrasound (TVUS), and serum estradiol levels. For example, it is known to adjust gonadotropin dosing during the stimulation period when one or both of the patient's serum estradiol level and number of follicles ≥12 mm are either too low or too high. Such an assessment and adjustment may be made at any time during the stimulation period, typically during a mid-follicular stage of stimulation, typically on the 5th or 6th or 7th day of stimulation. Thus, the treatment may comprise administering a daily dose of HP-hMG (such as MENOPUR®), at a starting daily dose from 75 to 450 IU/day, such as 150 IU/day, by injection from day 1 (stimulation day 1) to, e.g., at least day 5 (stimulation day 5) of treatment. Thereafter, the dose may be adjusted (e.g., depending on the patient's ovarian response) up or down (e.g., in increments of 75 IU hMG) to a maximum daily dose of 300 or 450 IU hMG or minimum daily dose of 75 IU hMG.

As noted above, HP-hMG administration continues daily until the desired level of follicle production is reached. For example, HP-hMG may be administered until three follicles have developed with a diameter of ≥17 mm, as may be determined by TVUS. Typically, the maximum HP-hMG dosing period is 20 days, with a typical dosing period of 8-12 days, more specifically typically about 9-11 days, including about 10 days.

In some embodiments, the treatment methods include the administration of a GnRH antagonist during a portion of the period of gonadotropin (e.g., HP-hMG) administration. For example, a GnRH antagonist may be administered once the lead follicle reaches 14 mm in diameter, and continued through the remainder of the period of gonadotropin (e.g., HP-hMG) administration. For example, a GnRH antagonist may be administered starting on the 5th or 6th or 7th day of stimulation (e.g., stimulation day 6), and continued through the remainder of the period of gonadotropin (e.g., HP-hMG) administration. When the GnRH antagonist is ganirelix acetate (such as ORGALUTRAN®), a typical dose is 0.25 mg/day administered subcutaneously.

In other embodiments, the treatment methods include the administration of a GnRH agonist prior to conducting controlled ovarian stimulation, such as the administration of triptorelin (typically at 0.1 mg/day subcutaneously) or leuprorelin (e.g., leuprolide acetate, e.g., LUPRON®) prior to conducting controlled ovarian stimulation.

In some embodiments, the treatment methods further comprise triggering final follicular maturation. For example, once the desired level of follicle production is reached, trigger of final follicular maturation can be stimulated by methods known in the art, such as by a bolus injection of human chorionic gonadotropin (hCG). For example, trigger of final follicular maturation may be stimulated in a patient with ≥3 follicles of ≥17 mm in diameter each, and, typically, estradiol (E2) levels <10,000 pmol/mL. Thus, in some embodiments, the treatment methods may comprise administering hCG to trigger final follicular maturation. The dose of hCG may be 5,000 IU to 10,000 IU. A typical dose of recombinant hCG (such as OVITRELLE®, Merck) is 250 μg (6,500 IU of hCG activity), usually administered by a single subcutaneous injection.

A GnRH agonist may be used as an alternative to use of hCG to trigger final follicular maturation. Thus, in some embodiments, the treatment methods may comprise administering a gonadotropin releasing hormone (GnRH agonist) to trigger final follicular maturation. A GnRH agonist may be used to trigger final follicular maturation, for example, in the event of excessive response, such as in a patient who, after COS treatment, has >25 follicles ≥12 mm in diameter or serum estradiol (E2) levels ≥5,000 pmol/L, or has >30 follicles ≥12 mm in diameter or serum estradiol (E2) levels ≥5,000 pmol/L, or estradiol (E2) levels ≥10,000 pmol/L, or ≥20 follicles ≥12 mm in diameter or estradiol (E2) levels ≥15,000 pmol/L. The GnRH agonist may be leuprolide acetate, e.g., LUPRON®, typically used at a dose of, e.g. 1-4 mg. The GnRH agonist may be triptorelin acetate, e.g., DECAPEPTYL®, typically used at a dose of, e.g., 0.2 mg. When a GnRH agonist is used to trigger final follicular maturation, a small amount of hCG also may be used, such as, for example 500-3000 IU hCG. When a GnRH agonist is used to trigger final follicular maturation, a “freeze all” protocol (discussed below) typically is followed, e.g., for safety reasons.

In some embodiments, the treatment methods further comprise retrieving oocytes and fertilizing the oocytes by methods known in the art, such as ICSI.

In some embodiments, the treatment method is a fresh transfer method. For fresh transfer methods, one or more blastocysts are selected for transfer. Remaining blastocysts can be frozen by methods known in the art for future transfer (including vitrification). Thus, in fresh transfer embodiments the methods comprise retrieving oocyte(s), fertilizing the oocyte(s), allowing the fertilized oocyte(s) to develop to the blastocyst stage, retrieving blastocyst(s), optionally selecting a blastocyst(s) based on assessment of quality/morphology, and implanting a fresh blastocyst (optionally selected based on, e.g., assessment of quality/morphology) in a uterus. In specific embodiments, the compositions and methods described herein are used in a single blastocyst transfer protocol, wherein a single blastocyst is selected for fresh transfer. In accordance with those embodiments, remaining blastocysts can be frozen by methods known in the art for future transfer.

In some embodiments, the method is a frozen transfer method. In frozen transfer embodiments, the methods comprise retrieving oocyte(s), fertilizing the oocyte(s), allowing the fertilized oocyte(s) to develop to the blastocyst stage, optionally assessing chromosomal quality of the blastocyst(s), freezing one or more or all blastocyst(s), and implanting a thawed-frozen blastocyst (e.g., a euploid blastocyst selected based on chromosomal assessment) in a uterus. For frozen and “freeze all” methods, selected blastocysts are frozen by methods known in the art for future implantation/transfer.

In some embodiments, unfertilized oocytes are frozen. In such embodiments, the methods comprise retrieving oocyte(s) and freezing one or more or all retrieved oocytes for future fertilization by methods known in the art. In such embodiments, the methods may subsequently comprise thawing one or more frozen oocytes, fertilizing the oocyte(s), allowing the fertilized oocyte(s) to develop to the blastocyst stage, optionally selecting blastocyst(s) based on assessment of quality/morphology, and implanting a blastocyst (optionally selected based on, e.g., visual assessment of quality/morphology) in a uterus. Alternatively, the methods may comprise retrieving oocyte(s), freezing one or more or all retrieved oocytes for future fertilization, subsequently thawing one or more frozen oocytes, fertilizing the oocyte(s), allowing the fertilized oocyte(s) to develop to the blastocyst stage, conducting chromosomal assessment of blastocyst(s), freezing blastocyst(s), and implanting a thawed-frozen blastocyst (e.g., a euploid blastocyst selected based on chromosomal assessment) in a uterus.

As noted above, in some embodiments, the methods comprise assessing chromosomal quality of the blastocyst(s) or selecting blastocyst(s) based on chromosomal assessment. This may be done by methods known in the art, such as Preimplantation Genetic Testing for Aneuploidy (PGT-A also known as PGS) or Preimplantation Genetic Diagnosis (PGD), which is used to test blastocysts (embryos) for genetic and chromosomal information. When PGS or PGD is used, all chromosomes can be assessed and only blastocysts identified at low risk of chromosome abnormalities are selected for embryo transfer (implantation in a uterus). This is an alternative to traditional methods where embryos are chosen according to their appearance under the microscope after three or five days of development in an incubator.

As set forth above, the methods described herein are useful for increasing ongoing pregnancy rates, as compared to comparable methods using recombinant follicle stimulating hormone (rFSH) as the gonadotropin. In particular, the methods described herein result in increased ongoing pregnancy rates, as compared to comparable methods using rFSH (e.g., GONAL-F) as the gonadotropin. As reported in Example 1, the methods described herein may result in a 15% or 19% or greater ongoing pregnancy rate.

Thus, according to some embodiments, there are provided assisted reproductive technology methods for treating a woman diagnosed with one or both of oligoovulation and PCOS and predicted to have a high ovarian response to controlled ovarian stimulation, comprising identifying a woman as diagnosed with one or both of oligoovulation and PCOS and as having a serum anti-Müllerian hormone (AMH) level 35.7±0.5 pmol/L (≥5.0±0.2 ng/ml), and conducting infertility treatment, e.g., controlled ovarian stimulation, by administering to the identified woman an amount of highly purified menotropin (HP-hMG) effective to stimulate follicle development. The methods may further comprise identifying the woman as having one or more of (i) a serum luteinizing hormone (LH) level of, e.g., greater than or equal to 7 U/L prior to treatment, (ii) a serum testosterone level of, e.g., greater than or equal to 1.10 nmol/L prior to treatment, and (iii) a serum estradiol level of, e.g., greater than or equal to 145 pmol/L prior to treatment. The HP-hMG may be administered at a dose of 75 to 450 IU hMG per day, such as at a dose of 150 IU hMG per day, typically from day 1 to at least, e.g., day 5 of treatment, at which time the dose may be adjusted up or down (e.g., in increments of 75 IU HP-hMG) depending on the patient's response, until the desired level of follicular maturation is reached for trigger of final follicular maturation. The methods may further comprise administering a gonadotropin-releasing hormone antagonist (GnRH antagonist) starting on, e.g., day 6 of treatment. The methods may further comprise triggering final follicular maturation by administering human chorionic gonadotropin (hCG) or a gonadotropin-releasing hormone agonist (GnRH agonist), optionally supplemented with hCG. As discussed above and shown in Example 1 below, the methods are effective to increase ongoing pregnancy rate after in vitro fertilization compared to treatment (e.g., controlled ovarian stimulation) by administration of recombinant follicle-stimulating hormone (rFSH).

In an example, the method comprises identifying a woman as diagnosed with one or both of oligoovulation and PCOS (including oligoovulation caused by PCOS) and as having a serum anti-Müllerian hormone (AMH) level 35.7±0.5 pmol/L (≥5.0±0.2 ng/ml), and conducting infertility treatment, e.g., controlled ovarian stimulation, by administering to the identified woman HP-hMG at a dose of, e.g., 150 IU per day, from day 1 of treatment to at least, e.g., day 5 of treatment, at which time the dose may be adjusted up or down (e.g., in increments of 75 IU HP-hMG) depending on the patient's response, until the desired level of follicular maturation is reached for trigger of final follicular maturation. In this example, the maximum daily dose would be 300 or 450 IU HP-hMG and the minimum daily dose would be 75 IU HP-hMG. The HP-hMG would be administered until the desired level of follicle production is reached, for a total treatment period (stimulation period) of from about 1 to about 20 days, typically for a total treatment (stimulation) period of from 8 to 12 days, more specifically typically about 9-11 days, including about 10 days.

According to the present invention there also is provided a composition (e.g., a pharmaceutical composition) comprising HP-hMG for use in the treatment of infertility in a patient (e.g., a woman) with (e.g., identified or diagnosed with) PCOS who has a serum AMH level≥35.7±0.5 pmol/L (≥5.0±0.2 ng/ml) prior to treatment/stimulation. (The patient is not an anovulatory patient.) The treatment of infertility may be treatment of infertility by controlled ovarian stimulation. The composition may comprise 75 to 450 IU HP-hMG. The treatment may comprise administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment. The daily dose of HP-hMG may be adjusted during the stimulation period, e.g., based on the patient's response, e.g., after treatment at the starting dose from day 1 to, e.g., day 5 of treatment, until the desired level of follicle production is reached, for a total treatment period (stimulation period) of from about 1 to about 20 days, typically for a total treatment (stimulation) period of from 8 to 12 days, more specifically typically about 9-11 days, including about 10 days.

According to the present invention there also is provided a composition (e.g., a pharmaceutical composition) comprising HP-hMG for use in the treatment of infertility in a patient (e.g., a woman) with (e.g., identified or diagnosed with) oligoovulation caused by PCOS who has a serum AMH level≥35.7±0.5 pmol/L (≥5.0±0.2 ng/ml) prior to treatment/stimulation. (The patient is not an anovulatory patient.) The treatment of infertility may be treatment of infertility by controlled ovarian stimulation. The composition may comprise 75 to 450 IU HP-hMG. The treatment may comprise administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment. The daily dose of HP-hMG may be adjusted during the stimulation period, e.g., based on the patient's response, e.g., after treatment at the starting dose from day 1 to, e.g., day 5 of treatment, until the desired level of follicle production is reached, for a total treatment period (stimulation period) of from about 1 to about 20 days, typically for a total treatment (stimulation) period of from 8 to 12 days, more specifically typically about 9-11 days, including about 10 days.

In any embodiments, the composition may be for treatment of a patient who has a serum AMH level≥35.7±0.5 pmol/L (≥5.0±0.2 ng/ml) prior to treatment/stimulation and a serum estradiol level of ≥145 pmol/L (e.g., a serum estradiol level of ≥150 pmol/L) prior to treatment/stimulation. Serum estradiol levels may be measured by methods well known in the art, as illustrated in Example 1. In any embodiments, the treatment may comprise a further step of identifying, prior to treatment/stimulation, a patient having a serum AMH level≥35.7±0.5 pmol/L (≥5.0±0.2 ng/ml), such as an AMH level≥35.7±0.5 pmol/L (≥5.0±0.2 ng/ml) when measured using a Beckmann-Coulter Gen 2 assay as described in Arce et al., Fertility and Sterility 99: 1644-53 (2013), or an equivalent AMH level assessed by a different method.

In any embodiments, the treatment may comprise a further step of identifying, prior to treatment/stimulation, a patient having a serum AMH level≥35.7±0.5 pmol/L (≥5.0±0.2 ng/ml) as disclosed above and a serum estradiol level of ≥145 pmol/L (e.g., a serum estradiol level of ≥150 pmol/L.

In any embodiments, the composition may be for treatment of a patient who has one or more of a serum luteinizing hormone (LH) level of ≥7 U/L (e.g., a serum luteinizing hormone of ≥7.55 U/L) prior to treatment/stimulation and/or a serum testosterone level of ≥1.10 nmol/L (e.g., a serum testosterone level of ≥1.14 nmol/L) prior to treatment/stimulation. Serum LH and testosterone levels may be measured by methods well known in the art, as illustrated in Example 1. Thus, in any embodiments the treatment may comprise a further step of identifying a patient having a serum luteinizing hormone (LH) level of ≥7 U/L (e.g., a serum luteinizing hormone of ≥7.55 U/L) prior to treatment/stimulation and/or a serum testosterone level of ≥1.10 nmol/L (e.g., a serum testosterone level of ≥1.14 nmol/L) prior to treatment/stimulation.

In any embodiments, the treatment may comprise identifying (diagnosing) a patient who has (a) a serum anti-Müllerian hormone (AMH) level≥35.7±0.5 pmol/L (≥5.0±0.2 ng/ml) prior to treatment/stimulation and (b) a serum estradiol level of ≥145 pmol/L (e.g., a serum estradiol level of ≥150 pmol/L) prior to treatment/stimulation, and optionally also has one or both of (c) a serum testosterone level of ≥1.10 nmol/L (e.g., a serum testosterone level of ≥1.14 nmol/L) prior to treatment/stimulation, and (d) a serum luteinizing hormone (LH) level of ≥7 U/L (e.g., a serum luteinizing hormone of ≥7.55 U/L) prior to treatment/stimulation; and administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

According to the present invention there is also provided a composition (e.g., a pharmaceutical composition) comprising HP-hMG for use in the treatment of infertility in a patient (e.g., a woman) with (e.g., identified or diagnosed with) PCOS (a non-anovulatory patient) and who has a serum AMH level≥35.7±0.5 pmol/L (≥5.0±0.2 ng/ml) prior to treatment/stimulation, the treatment comprising:
identifying (e.g., diagnosing) a patient with PCOS who has a serum AMH level≥35.7±0.5 pmol/L (≥5.0±0.2 ng/ml), such as when measured using a Beckmann-Coulter Gen 2 assay as described in Arce et al., Fertility and Sterility 99: 1644-53 (2013), or an equivalent AMH level assessed by a different method, prior to treatment/stimulation; and
administering (to the patient) a daily dose of HP-hMG of from 75 to 450 IU per day, preferably from 75-225 IU/day, more preferably 150 or 225 IU/day, most preferably 150 IU/day, optionally from day 1 of treatment to at least day 5 of treatment. The daily dose may be adjusted during the stimulation period, e.g., based on the patient's response, e.g., after treatment at the starting dose from day 1 to, e.g., day 5 of treatment, until the desired level of follicle production is reached, for a total stimulation period (treatment period) of from about 1 to about 20 days, typically for a total stimulation period of from 8 to 12 days, more specifically typically about 9-11 days, including about 10 days. The treatment of infertility may be treatment of infertility by controlled ovarian stimulation. The composition may comprise 75 to 450 IU HP-hMG.

According to the present invention there is also provided a composition (e.g., a pharmaceutical composition) comprising HP-hMG for use in the treatment of infertility in a patient (e.g., a woman) with (e.g., identified or diagnosed with) oligoovulation caused by PCOS (a non-anovulatory patient) and who has a serum AMH level≥35.7±0.5 pmol/L (≥5.0±0.2 ng/ml) prior to treatment/stimulation, the treatment comprising:
identifying (e.g., diagnosing) a patient with oligoovulation caused by PCOS who has a serum AMH level≥35.7±0.5 pmol/L (≥5.0±0.2 ng/ml), such as when measured using a Beckmann-Coulter Gen 2 assay as described in Arce et al., Fertility and Sterility 99: 1644-53 (2013), or an equivalent AMH level assessed by a different method, prior to treatment/stimulation; and
administering (to the patient) a daily dose of HP-hMG of from 75 to 450 IU per day, preferably from 75-225 IU/day, more preferably 150 or 225 IU/day, most preferably 150 IU/day, optionally from day 1 of treatment to at least day 5 of treatment. The daily dose may be adjusted during the stimulation period, e.g., based on the patient's response, e.g., after treatment at the starting dose from day 1 to, e.g., day 5 of treatment, until the desired level of follicle production is reached, for a total stimulation period (treatment period) of from about 1 to about 20 days, typically for a total stimulation period of from 8 to 12 days, more specifically typically about 9-11 days, including about 10 days. The treatment of infertility may be treatment of infertility by controlled ovarian stimulation. The composition may comprise 75 to 450 IU HP-hMG.

In any embodiments, the treatment may comprise identifying (e.g., diagnosing) a patient who has (a) a serum anti-Müllerian hormone (AMH) level≥35.7±0.5 pmol/L (≥5.0±0.2 ng/ml) prior to treatment/stimulation and (b) a serum estradiol level of ≥145 pmol/L (e.g., a serum estradiol level of ≥150 mon) prior to treatment/stimulation, and optionally also has one or both of (c) serum testosterone level of ≥1.10 nmol/L (e.g., a serum testosterone level of ≥1.14 nmol/L) prior to treatment/stimulation, and (d) a serum luteinizing hormone (LH) level of ≥7 U/L (e.g., a serum luteinizing hormone of ≥7.55 U/L) prior to treatment/stimulation; and administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

According to the present invention in a further aspect there is provided the use of HP-hMG in the manufacture of a medicament (e.g., a pharmaceutical composition) for the treatment of infertility in a patient (e.g., a woman) with (e.g., identified or diagnosed with) oligoovulation and/or PCOS (including a woman with oligoovulation caused by PCOS) (e.g., a non-anovulatory woman) who has a serum AMH level≥35.7±0.5 pmol/L (≥5.0±0.2 ng/ml) prior to treatment/stimulation. The treatment of infertility may be treatment of infertility by controlled ovarian stimulation. The composition may comprise 75 to 450 IU HP-hMG. The treatment may comprise identifying a patient having a serum anti-Müllerian hormone (AMH) level≥35.7±0.5 pmol/L (≥5.0±0.2 ng/ml) prior to treatment/stimulation (e.g. when measured using a Beckmann-Coulter Gen 2 assay as described in Arce et al., Fertility and Sterility 99: 1644-53 (2013), or an equivalent AMH level assessed by a different method). The treatment may comprise a further step of identifying, prior to treatment/stimulation, a patient having a serum estradiol level of ≥145 pmol/L (e.g., a serum estradiol level of ≥150 pmol/L. The treatment may additionally comprise a further step of identifying a patient having a serum luteinizing hormone (LH) level of ≥7 U/L (e.g., a serum luteinizing hormone of ≥7.55 Li) prior to treatment/stimulation and/or a serum testosterone level of ≥1.10 nmol/L (e.g., a serum testosterone level of ≥7_0.14 nmol/L) prior to treatment/stimulation. The treatment of infertility may comprise administering HP-hMG at a dose of 75 to 450 IU hMG per day until the desired level of follicle production is reached.

As set forth above, the treatment of infertility as disclosed herein, i.e., in accordance with each of the various embodiments disclosed herein, is associated with a higher ongoing pregnancy rate compared to a comparable method of treatment using recombinant follicle-stimulating hormone (rFSH) as the gonadotropin.

As noted above, in any embodiments the composition may comprise 75 to 450 IU HP-hMG, such as MENOPUR®.

As noted above, in any embodiments, the treatment of infertility may comprise administering (to the patient) a dose of 75 to 450 IU HP-hMG per day, including 75 IU/day, 150 IU/day, 225 IU/day, 300 IU/day, 375 IU/day or 450 IU/day (which may be adjusted during the stimulation period, e.g., based on the patient's response, e.g., after treatment at the starting dose from day 1 to, e.g., day 5 of treatment), until the desired level of follicle production is reached, for a total stimulation period (treatment period) of from about 1 to about 20 days, typically for a total stimulation period of from 8 to 12 days, more specifically typically about 9-11 days, including about 10 days. Thus, in any embodiments, the treatment may comprise administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

In an example, the treatment comprises administering (to the patient) a dose of 150 IU HP-hMG per day from day 1 to at least day 5 of treatment. The dose may be adjusted (e.g. depending on patient's response) up or down from, e.g., day 6 of treatment (e.g., in increments of 75 IU hMG). In this example, the maximum daily dose is 300 or 450 IU hMG and the minimum daily dose is 75 IU hMG.

In any embodiments, the treatment may comprise a further step of administering a GnRH antagonist starting once the lead follicle reaches 14 mm in diameter and/or on the 5th or 6th or 7^{th} day of stimulation (e.g., stimulation day 6), and continued through the remainder of the period of HP-hMG administration.

In any embodiments, the treatment may further comprise triggering final follicular maturation, as described above. Thus, the treatment may comprise administering hCG (e.g. recombinant hCG) or a GnRH agonist to trigger final follicular maturation. As discussed above, when a GnRH agonist is used to trigger final follicular maturation, a small amount of hCG also may be used.

In any embodiments, the treatment may further comprise retrieving (e.g. harvesting) oocyte(s); fertilizing (e.g. inseminating) the oocytes (s); and allowing the fertilized oocytes to develop to the blastocyst stage. The fertilization (e.g. insemination) may be in vitro fertilization, optionally intra-cytoplasmic sperm injection (ICSI).

In any embodiments, the treatment may be a fresh transfer method comprising retrieving oocyte(s), fertilizing the oocyte(s), allowing the fertilized oocyte(s) to develop to the blastocyst stage, retrieving blastocyst(s), optionally selecting a blastocyst(s) based on assessment of quality/morphology, and implanting a fresh blastocyst (optionally selected based on, e.g., assessment of quality/morphology) in a uterus. The treatment may be a single blastocyst transfer protocol, wherein a single blastocyst is selected for fresh transfer. Optionally, remaining blastocysts can be frozen by methods known in the art for future transfer.

In any embodiments, the treatment may be a frozen transfer method comprising retrieving oocyte(s), fertilizing the oocyte(s), allowing the fertilized oocyte(s) to develop to the blastocyst stage, optionally assessing chromosomal quality of the blastocyst(s), freezing one or more or all blastocyst(s), and implanting a thawed-frozen blastocyst (e.g., a euploid blastocyst selected based on chromosomal assessment) in a uterus. For frozen and “freeze all” methods, selected blastocysts are frozen by methods known in the art for future implantation/transfer.

In any embodiments, the method may involve freezing unfertilized oocytes. Thus, the methods may comprise retrieving oocyte(s), freezing one or more or all retrieved oocytes, subsequently thawing one or more frozen oocytes, fertilizing the oocyte(s), allowing the fertilized oocyte(s) to develop to the blastocyst stage, optionally selecting blastocyst(s) based on assessment of quality/morphology, and implanting a blastocyst (optionally selected based on, e.g., visual assessment of quality/morphology) in a uterus. Alternatively, the methods may comprise retrieving oocyte(s), freezing one or more or all retrieved oocytes, subsequently thawing one or more frozen oocytes, fertilizing the oocyte(s), allowing the fertilized oocyte(s) to develop to the blastocyst stage, conducting chromosomal assessment of blastocyst(s), freezing blastocyst(s), and implanting a thawed-frozen blastocyst (e.g., a euploid blastocyst selected based on chromosomal assessment) in a uterus.

Further aspects of the methods described herein are illustrated in the following examples, which are not limiting in any respect.

### EXAMPLES

### Example 1— MEGASET HR Clinical Trial and Retrospective Analysis

The following describes a retrospective analysis of data collected in a multicenter, randomized, assessor-blind, controlled non-inferiority trial in 620 women, 21-35 years, with BMI 18-30 kg/m² and serum anti-Müllerian hormone (AMID ≥35.7 pmol/L undergoing intracytoplasmic sperm injection and single blastocyst transfer (fresh transfer). The trial was titled “MENOPUR® in a Gonadotropin-Releasing Hormone (GnRH) Antagonist Cycle With Single-Blastocyst Transfer in a High Responder Subject Population (MEGASET HR)” (ClinicalTrials.gov identifier NCT02554279). Further details can be found at clinicaltrials.gov/ct2/show/record/NCT02554279 and in Witz et al., Fertility and Sterility, in press (published on line Mar. 29, 2020).

### 1. Study Population

The main inclusion criteria were for females aged 21 to 35 years with regular ovulatory menstrual cycles of 21 to 45 days, with a Body Mass Index (BMI) between 18 and 30 kg/m² who desire pregnancy. The patients/subjects were predicted-high responders, which was defined as subjects who have a serum anti-Müllerian hormone (AMH) ≥5 ng/mL (35.71 pmol/L) at screening. The subjects had a documented history of infertility (e.g., unable to conceive for at least 12 months or for at least 6 months if receiving donor sperm) with a menstrual cycle day 2 or day 3 serum FSH level between 1 and 12 IU/L (inclusive).

The exclusion criteria were known stage III-IV endometriosis; history of recurrent miscarriage not followed by a live birth (with recurrent defined as two or more consecutive miscarriages); and previous in vitro fertilization (IVF) or assisted reproductive technology (ART) failure due to a poor response to gonadotropins (with poor response defined as development of ≤2 mature follicles or history of 2 previous failed cycle cancellations prior to oocytes retrieval due to poor response). Anovulatory women also were excluded.

### 2. Study Protocol

This was a multicenter, randomized, assessor-blind phase IV clinical trial comparing HP-hMG and rFSH in a GnRH antagonist cycle with compulsory single-blastocyst transfer (fresh transfer) in a high responder subject population in the United States. The aim of this study was to demonstrate that HP-hMG is at least non-inferior to rFSH with respect to ongoing pregnancy rate (OPR) in potential high-responders undergoing IVF/ICSI treatment.

Subjects were classified as potential high ovarian responders based on a serum level of AMH ≥5.0 ng/ml (e.g. ≥35.7 pmol/L) by the Beckmann-Coulter Gen 2 assay as described in Arce et al., Fertility and Sterility 99: 1644-53 (2013), using a single reference laboratory (ReproSource, Inc., Woburn, Mass.) utilizing materials and reagents from the Beckman Coulter-DSL assay (Chaska, Minn.).

Subjects were randomized 1:1 to undergo COS with either a 150 IU dose of HP-hMG (N=311; MENOPUR®, Ferring Pharmaceuticals, Inc.) or rFSH (N=309; GONAL-F, EMD Serono) as the gonadotropin in a GnRH antagonist cycle. Treatment was initiated on day 2 or 3 of the menstrual cycle at a dose of 150 IU HP-hMG or rFSH for the first 5 days. From Stimulation day 6 onward, dosing could be adjusted every day as needed by 75 IU per adjustment, based on follicular response assessed by TVUS. However, the maximum gonadotropin dose was 300 IU/day. Gonadotropin dosing could continue for a maximum of 20 days and coasting was prohibited.

When the lead follicle was >14 mm in diameter, a GnRH antagonist (ganirelix acetate) was initiated at a daily dose of 0.25 mg and continued throughout the gonadotropin treatment period.

A single injection of 250 μg hCG (choriogonadotropin alfa) was administered to induce final follicular maturation as soon as 3 follicles of ≥17 mm diameter were observed on TVUS. However, if a subject had excessive ovarian response (>30 follicles of ≥12 mm each and/or estradiol (E2) levels ≥5,000 pg/mL), a GnRH agonist (4 mg leuprolide acetate) was administered ≥12 hours after the last GnRH antagonist dose, fresh transfer was canceled, all blastocysts were biopsied; and viable blastocysts were frozen for use in a subsequent transfer cycle in order to decrease risk of OHSS.

Oocyte retrieval took place roughly 36 hours after hCG or GnRH agonist administration. Oocytes were inseminated using partner sperm by ICSI 4±1 hours after retrieval. Oocyte, embryo and blastocyst quality were assessed. On Day 5 following ICSI, a single blastocyst of the best quality by morphology (Gardner and Schoolcraft scale) was transferred (fresh transfer); all remaining blastocysts were frozen using the vitrification method.

The day after oocyte retrieval, vaginal progesterone inserts (100 mg twice a day—ENDOMETRIN®; Ferring) were initiated for luteal phase support and this continued until the day of the β-hCG test (10 to 15 days after blastocyst/embryo transfer). Luteal support could be continued until ongoing pregnancy was confirmed.

Biochemical pregnancy was confirmed by a positive β-hCG test approximately 2 weeks after blastocyst transfer. Clinical pregnancy was confirmed by TVUS indicating at least one intrauterine gestational sac with fetal heart beat at 5 to 6 weeks gestation. Ongoing pregnancy was confirmed by at least one intrauterine viable fetus at 10 to 11 weeks gestation.

For subjects with no ongoing pregnancy in the fresh cycle, single frozen blastocyst transfers could be initiated within 6 months of the subject's randomization in the trial. PGS results could be used to select euploid blastocysts for frozen transfer. Frozen-thawed embryo transfer cycle data was collected, including blastocyst transfer information, β-hCG test, clinical pregnancy, ongoing pregnancy, pregnancy loss rate and live birth.

Post-trial follow-up included collection of delivery information (live birth and neonatal health), which was collected for all subjects with an ongoing pregnancy in the fresh cycle or the 1-year post-randomization frozen-thawed embryo replacement cycles. Live birth rate after the fresh cycle and cumulative live birth rate after fresh and 6-month post-randomization frozen-thawed embryo replacement cycles were evaluated as part of the post-trial follow-up.

The HP-hMG used was MENOPUR® (provided by Ferring Pharmaceuticals, Inc.), provided as a vial containing dry HP-hMG (75 IU HP-hMG, providing 75 IU FSH activity and 75 IU LH activity, including LH activity provided by hCG) and vials containing solvent for reconstitution. After reconstitution, each vial contains 75 IU of FSH activity and 75 IU of LH activity, including LH activity provided by hCG.

The FSH used was recombinant FSH (GONAL-F, EMD Serono), provided as solution for injection.

The other drugs used were:
Ganirelix Acetate Injection, manufactured by Merck, provided as a pre-filled syringe (0.5 mL) delivering 0.25 mg ganirelix. Once the lead follicle measures ≥14 mm and/or serum E2 levels are ≥300 pg/mL, ganirelix acetate was initiated at a daily dose of 0.25 mg and continued throughout the gonadotropin treatment period.
OVIDREL® (choriogonadotropin alfa), manufactured by EMD Serono, provided as a pre-filled syringe (0.5 mL) delivering 250 μg choriogonadotropin alfa, administered as a single injection as soon as 3 follicles of ≥17 mm diameter were observed on TVUS.
ENDOMETRIN® (progesterone), manufactured by Ferring, provided as inserts to be administered vaginally 2 times daily, each delivering 100 mg (200 mg/day).

The primary end point was ongoing pregnancy rate, with ongoing pregnancy defined as presence of at least one intrauterine pregnancy with a viable fetus with a detectable fetal heartbeat at 10-11 weeks gestation. Secondary endpoints included:
biochemical pregnancy rate (positive β-hCG test)
clinical pregnancy rate (transvaginal ultrasonography showing at least one intrauterine gestational sac with fetal heart beat at 5-6 weeks gestation)
early pregnancy loss (defined as 2 positive β-hCG tests but no ongoing pregnancy at 10-11 weeks gestation in the fresh cycle.)
live birth rate
follicular development as assessed by TVUS, follicle level (total number of follicles, number of follicles ≤9 mm, 10-11 mm, 12-14 mm, 15-16 mm, and ≥17 mm) and subject level (largest follicle size, average follicle size, average size of 3 largest follicles, and average number of follicles ≥17 mm, ≥15 mm, and ≥12 mm)
endocrine profile (serum estradiol [E2], progesterone [P4], hCG, LH)
oocytes retrieved, fertilization rate, and embryo quality

### 3. Serum Assays

Blood samples were taken prior to and throughout the stimulation period, including prior to start of stimulation, on stimulation day 6, and on the last day of stimulation. Serum was analyzed using ELISA for AMH (Beckman Coulter Gen 2), FSH, LH, and hCG, using two dimensional high performance liquid chromatography with tandem mass spectrometry for estradiol and using liquid chromatography with tandem mass spectrometry for progesterone and testosterone. The lower detection limits were as follows: FSH 0.017mIU/mL; LH 0.005 mIU/mL; βhCG 0.5 mIU/mL; estradiol 1.0 pgl/mL, progesterone 10 ng/dL, and testosterone 2.5 ng/dL.

### 4. Results and Retrospective Analysis

The non-inferiority objective for the primary endpoint of ongoing pregnancy was met. HP-hMG was associated with numerically higher ongoing pregnancy rates vs rFSH (35.5% vs 30.7%, P>0.05). The average number of oocytes per patient (±SD) in the rFSH arm (22.2±11.54) was higher than in the hMG arm (15.1±10.12), a difference in ovarian response that was accompanied by statistically significant increases in rates of OHSS (21.4% vs 9.7%; p<0.05).

Retrospective analyses in the modified intent-to-treat population (all randomized subjects who received at least 1 dose of gonadotropin) included assessment of primary endpoint rate by infertility diagnoses. The retrospective analysis by infertility diagnosis showed no significant differences in ongoing pregnancy rate between treatment groups in those diagnosed with endometriosis, male factor, tubal infertility, idiopathic, or other. However, among those diagnosed with oligoovulation, HP-hMG treatment (N=50) was unexpectedly associated with a 19.2% higher ongoing pregnancy rate (95% confidence interval 1.2%, 37.3%) than rFSH treatment (N=56), with ongoing pregnancy rates of 46.0% vs. 26.8%, respectively.

As shown in the tables below, relative to the rest of the trial population, those with oligoovulation had higher mean baseline AMH (60.95 vs. 52.10 pmol/L, p<0.001), luteinizing hormone (7.55 vs. 6.45 U/L, p=0.007), testosterone (1.13 vs. 1.00 nmol/L, p=0.006), and estradiol (167.04 vs. 135.46 pmol/L, p=0.001) although FSH and BMI were similar. Comparisons between populations with and without oligoovulation were made using either t-tests (continuous parameters) or Fisher's exact test (categorical parameters).

Subject demographics:

| | Oligoovulation | Others | |
|---|---|---|---|
| Parameter | (n = 106) | (n = 154) | p-Value |
| Age, years | 29.65 ± 3.32 | 30.36 ± 2.98 | 0.030 |
| BMI, kg/m² | 24.78 ± 3.25 | 24.26 ± 3.35 | 0.138 |
| AMH, pmol/L* | 60.95 ± 29.50 | 52.10 ± 19.17 | <0.001 |
| FSH, U/L | 6.14 ± 1.57 | 6.32 ± 1.54 | 0.287 |
| LH, U/L | 7.55 ± 4.81 | 6.45 ± 3.50 | 0.007 |
| Testosterone, nmol/L | 1.14 ± 0.66 | 1.00 ± 0.42 | 0.006 |
| Estradiol, pmol/L | 167.04 ± 126.41 | 135.46 ± 61.94 | 0.001 |
| Progesterone, nmol/L | 0.97 ± 3.48 | 0.57 ± 1.77 | 0.087 |

Based on the elevated serum AMH, LH, testosterone, and estradiol levels (and a trend towards progesterone elevation), it is likely the oligoovulation patient population included patients with PCOS, e.g., patients whose oligoovulation was due to PCOS. This is because elevated AMH, LH, testosterone, estradiol, and progesterone levels are hallmarks of PCOS, and because other common causes of oligoovulation (such as ovarian failure, hyperprolactinemia, thyroid dysfunction and adrenal dysfunction were likely to have been excluded by the study inclusion and exclusion criteria).

Subject responses by cause of infertility:

| | Oligoovulation | Others | |
|---|---|---|---|
| Parameter | (n = 106) | (n = 154) | p-Value |
| Day 6, Estradiol | 2363.9 ± 1864.4 | 2327.7 ± 2145.5 | 0.881 |
| pmol/L | | | |
| Day 6, Progesterone | 0.58 ± 0.83 | 0.55 ± 0.84 | 0.749 |
| nmol/L | | | |
| Trigger day, | 10089 ± 5483.7 | 11237 ± 7258.7 | 0.152 |
| Estradiol pmol/L | | | |
| Trigger day, | 2.81 ± 3.52 | 2.74 ± 2.81 | 0.824 |
| Progesterone nmol/L | | | |
| Oocytes retrieved | 17.95 ± 10.48 | 18.91 ± 11.61 | 0.436 |
| Early OHSS n (%) | 16 (15.1%) | 57 (11.1%) | 0.248 |

Subject responses by treatment:

| | Oligoovulation | Others | |
|---|---|---|---|
| Parameter | (n = 106) | (n = 154) | p-Value |
| Day 6, Estradiol | 1539.1 ± 1485.3 | 3026.9 ± 1885.5 | <0.001 |
| pmol/L | | | |
| Day 6, Progesterone | 0.38 ± 0.62 | 0.75 ± 0.96 | 0.026 |
| nmol/L | | | |
| End of stim, | 9910.7 ± 4986.5 | 10253 ± 5951.2 | 0.767 |
| Estradiol pmol/L | | | |
| End of stim, | 1.91 ± 2.86 | 3.64 ± 3.87 | 0.012 |
| Progesterone nmol/L | | | |
| Oocytes retrieved | 13.69 ± 9.14 | 21.75 ± 10.21 | <0.001 |
| Early OHSS n (%) | 6 (12.0%) | 10 (17.9%) | 0.430 |

Thus, the present inventor surprisingly found that patients predicted to be high-responders who are diagnosed with oligoovulation (including oligoovulation caused by PCOS) and treated with HP-hMG as the gonadotropin for COS (N=50) had a 19.2% higher ongoing pregnancy rate after fresh transfer (95% confidence interval 1.2%-37.3%) compared to those treated with rFSH as the gonadotropin for COS (N=56). This is greater than the improvement in ongoing pregnancy rate associated with HP-hMG treatment (with reference to FSH) in the total population of predicted high responders (35.5% vs 30.7%, P>0.05).

Thus, the present inventor found that selection of predicted high responder patients diagnosed with oligoovulation (including oligoovulation caused by PCOS) for treatment with hMG as the gonadotropin for COS, rather than FSH, may be associated with higher ongoing pregnancy rate. Accordingly, the invention described herein pertains to subjects selected pursuant to multiple criteria, including one or more or all of oligoovulation diagnosis; PCOS diagnosis; serum level of AMH ≥35.7±0.5 pmol/L (≥5.0±0.2 ng/ml) by the Beckmann-Coulter Gen 2 assay as described in Arce et al., Fertility and Sterility 99: 1644-53 (2013), or an equivalent serum AMH level determined by a different method; baseline serum estradiol ≥145 pmol/L, baseline serum LH≥7 U/L, and baseline serum testosterone ≥1.10 nmol/L.

### Example 2

An exemplary method of an infertility treatment for oligoovulation and/or PCOS patients (including oligoovulation caused by PCOS) who are predicted-high responders, is outlined below. The infertility treatment comprising COS with HP-hMG, rather than FSH, as the gonadotropin is associated with higher ongoing pregnancy rate.

Typically, a medical practitioner will oversee the treatment of infertility. Typically, the patient will be, or will have been, diagnosed with oligoovulation and/or PCOS (including oligoovulation caused by PCOS) by a medical practitioner. The patient may also be, or have been, diagnosed as a predicted-high responder, such as by a serum AMH test, for example based on having a serum level of AMH ≥35.7±0.5 pmol/L (≥5.0±0.2 ng/ml) by the Beckmann-Coulter Gen 2 assay as described in Arce et al., Fertility and Sterility 99: 1644-53 (2013), or an equivalent serum AMH level determined by a different method. Additionally or alternatively, the patient may be, or may have been, identified as having one or more of (i) a serum luteinizing hormone (LH) level of greater than or equal to 7 U/L prior to controlled ovarian stimulation, (ii) a serum testosterone level of greater than or equal to 1.10 nmol/L prior to controlled ovarian stimulation, and (iii) a serum estradiol level of greater than or equal to 145 pmol/L prior to controlled ovarian stimulation.

A patient diagnosed as having oligoovulation and/or PCOS (including oligoovulation caused by PCOS), and having (optionally identified as having) a serum level of AMH ≥35.7±0.5 pmol/L or ≥5.0±0.2 ng/ml prior to treatment), and optionally identified as having, one or more of (i) a serum luteinizing hormone (LH) level of greater than or equal to 7 U/L prior to controlled ovarian stimulation, (ii) a serum testosterone level of greater than or equal to 1.10 nmol/L prior to controlled ovarian stimulation, and (iii) a serum estradiol level of greater than or equal to 145 pmol/L prior to controlled ovarian stimulation, is selected for COS using HP-hMG (for example MENOPUR®, available from Ferring Pharmaceuticals) as the gonadotropin. As noted above, as reconstituted for use, each vial of MENOPUR® contains 75 IU FSH activity and 75 IU LH activity, including hCG-driven LH activity.

Controlled ovarian stimulation is begun (“stimulation day 1”) on day 2 or 3 of the patient's menstrual cycle. The treatment comprises administering a daily dose of MENOPUR®, such as 150 IU/day, by injection from day 1 (stimulation day 1) to at least day 5 (stimulation day 5) of treatment. The dose may be adjusted (e.g., depending on the patient's ovarian response) up or down (e.g., in increments of 75 IU hMG) to a maximum daily dose of 300 or 450 IU hMG or minimum daily dose of 75 IU hMG. The treatment may continue for up to 20 days (up to and including stimulation day 20), but typically is for 8-12 days, including about 10 days.

When the lead follicle is >14 mm in diameter, as assessed by TVUS, a GnRH antagonist (ganirelix acetate) may be initiated at a daily dose of 0.25 mg and continued throughout the gonadotropin stimulation treatment period.

Final follicular maturation is triggered with hCG or a GnRH agonist. A single injection of 250 μg hCG (choriogonadotropin alfa) may be administered to induce final follicular maturation as soon as 3 follicles of ≥17 mm diameter are observed on TVUS. Alternatively, a GnRH agonist may be used to trigger final follicular maturation, such as in the event of excessive response to COS, such as in a patient who, following COS treatment, has >30 follicles of ≥12 mm diameter or serum estradiol (E2) levels ≥5,000 pg/ml. When a GnRH agonist is used, it may be, e.g., leuprolide acetate, e.g., LUPRON®, at a dose of, e.g., 1-4 mg.

The method further comprises oocyte retrieval (generally roughly 36 hours after triggering final follicular maturation), fertilization, and subsequent procedures including retrieving blastocyst(s), and implanting a fresh blastocyst in a uterus, in accordance with the protocols described above and variations thereof that are known in the art.

What is claimed is:

1. A composition comprising highly purified menotropin (HP-hMG) for use in the treatment of infertility, optionally by controlled ovarian stimulation, in a patient with polycystic ovary syndrome (PCOS), wherein the patient has a serum anti-Müllerian hormone (AMH) level≥35.7±0.5 pmol/L (≥5.0±0.2 ng/ml) prior to treatment/stimulation.

2. A composition comprising highly purified menotropin (HP-hMG) for use in the treatment of infertility, optionally by controlled ovarian stimulation, in a patient with oligoovulation caused by polycystic ovary syndrome (PCOS), wherein the patient has a serum anti-Müllerian hormone (AMH) level≥35.7±0.5 pmol/L (≥5.0±0.2 ng/ml) prior to treatment/stimulation.

3. A composition for use according to claim 1 or claim 2, comprising 75 to 450 IU HP-hMG.

4. A composition for use according to any preceding claim, wherein the treatment of infertility comprises administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

5. A composition for use according to any preceding claim, wherein the treatment of infertility comprises:
identifying (e.g., diagnosing) a patient who has (a) a serum anti-Müllerian hormone (AMH) level≥35.7±0.5 pmol/L (≥5.0±0.2 ng/ml) prior to treatment/stimulation and (b) a serum estradiol level of ≥145 pmol/L (e.g., a serum estradiol level of ≥150 pmol/L) prior to treatment/stimulation, and optionally also has one or both of (c) a serum testosterone level of ≥1.10 nmol/L (e.g., a serum testosterone level of ≥1.14 nmol/L) prior to treatment/stimulation, and (d) a serum luteinizing hormone (LH) level of ≥7 U/L (e.g., a serum luteinizing hormone of ≥7.55 U/L) prior to treatment/stimulation; and
administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

6. A composition comprising highly purified menotropin (HP-hMG) for use in the treatment of infertility, optionally by controlled ovarian stimulation, in a patient with polycystic ovary syndrome (PCOS) and a serum anti-Müllerian hormone (AMH) level≥35.7±0.5 pmol/L (≥5.0±0.2 ng/ml) prior to treatment/stimulation, the treatment comprising:
identifying (e.g., diagnosing) a patient with PCOS who has a serum anti-Müllerian hormone (AMH) level≥35.7±0.5 pmol/L (≥5.0±0.2 ng/ml) prior to treatment/stimulation; and
administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

7. A composition comprising highly purified menotropin (HP-hMG) for use in the treatment of infertility, optionally by controlled ovarian stimulation, in a patient with oligoovulation caused by polycystic ovary syndrome (PCOS) and a serum anti-Müllerian hormone (AMH) level≥35.7±0.5 pmol/L (≥5.0±0.2 ng/ml) prior to treatment/stimulation, the treatment comprising:
identifying (e.g., diagnosing) a patient with oligoovulation caused by PCOS who has a serum anti-Müllerian hormone (AMH) level≥35.7±0.5 pmol/L (≥5.0±0.2 ng/ml) prior to treatment/stimulation; and
administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

8. A composition for use according to claim 6 or 7, wherein the treatment of infertility comprises:
identifying (e.g., diagnosing) a patient who has (a) a serum anti-Müllerian hormone (AMH) level≥35.7±0.5 pmol/L (≥5.0±0.2 ng/ml) prior to treatment/stimulation and (b) a serum estradiol level of ≥145 pmol/L (e.g., a serum estradiol level of ≥150 pmol/L) prior to treatment/stimulation, and optionally also has one or both of (c) a serum testosterone level of ≥1.10 nmol/L (e.g., a serum testosterone level of ≥1.14 nmol/L) prior to treatment/stimulation, and (d) a serum luteinizing hormone (LH) level of ≥7 U/L (e.g., serum luteinizing hormone of ≥7.55 U/L) prior to treatment/stimulation; and
administering a daily dose of HP-hMG to the patient of from 75-450 IU/day, preferably from 75-225 IU/day, more preferably 150 or 225 IU/day, most preferably 150 IU/day, optionally from day 1 of treatment to at least day 5 of treatment.

9. A composition for use according to any preceding claim, wherein the treatment of infertility increases ongoing pregnancy rate compared to treatment with recombinant follicle-stimulating hormone (rFSH).

10. A composition for use according to any preceding claim, wherein the treatment further comprises triggering final follicular maturation by administering hCG or a GnRH agonist, optionally supplemented with hCG.

11. A composition for use according to any preceding claim, wherein the treatment is a fresh transfer method further comprising retrieving oocyte(s), fertilizing the oocyte(s), allowing the fertilized oocyte(s) to develop to the blastocyst stage, optionally assessing the quality/morphology of the blastocyst(s), and implanting a fresh blastocyst (optionally selected based on, e.g., visual assessment of quality/morphology) in a uterus.

12. A composition for use according to any one of claims 1-10, wherein the treatment is a frozen transfer method further comprising retrieving oocyte(s), fertilizing the oocyte(s), allowing the fertilized oocyte(s) to develop to the blastocyst stage, optionally assessing the chromosomal quality of the blastocyst(s), freezing one or more or all blastocyst(s), and implanting a thawed frozen blastocyst (e.g., a euploid blastocyst selected based on chromosomal assessment) in a uterus.

13. A composition for use according to any one of claims 1-10, wherein the treatment further comprises:
retrieving oocyte(s), freezing unfertilized oocytes(s), subsequently thawing one or more oocyte(s), fertilizing one or more or all thawed oocyte(s), allowing fertilized oocyte(s) to develop to the blastocyst stage, optionally assessing the quality/morphology of the blastocyst(s), and implanting a blastocyst (optionally selected based on, e.g., visual assessment of quality/morphology) in a uterus; or
retrieving oocyte(s), freezing unfertilized oocytes(s), subsequently thawing one or more frozen oocytes, fertilizing one or more or all thawed oocyte(s), allowing fertilized oocyte(s) to develop to the blastocyst stage, optionally assessing chromosomal quality of the blastocyst(s), freezing one or more or all blastocyst(s), and implanting a thawed-frozen blastocyst (e.g., a euploid blastocyst selected based on chromosomal assessment) in a uterus.

14. A composition for use according to any preceding claim, further comprising a step of administering a GnRH antagonist starting on day 6 of treatment.

15. A composition for use according to any preceding claim, wherein the patient is not anovulatory, is 21-35 years old, and has a BMI of 18-30 kg/m² at the start of treatment.

16. An assisted reproductive technology method for treating a woman diagnosed with one or both of oligoovulation and PCOS and predicted to have a high ovarian response to controlled ovarian stimulation, comprising:
identifying a woman as diagnosed with one or both of oligoovulation and PCOS and as having a serum anti-Müllerian hormone (AMH) level 35.7±0.5 pmol/L (≥5.0±0.2 ng/ml) prior to treatment, and
conducting controlled ovarian stimulation by administering to the identified woman an amount of highly purified menotropin (HP-hMG) effective to stimulate follicle development.

17. The method of claim 16, further comprising identifying the woman as having one or more of (i) a serum luteinizing hormone (LH) level of greater than or equal to 7 U/L prior to treatment/stimulation, (ii) a serum testosterone level of greater than or equal to 1.10 nmol/L prior to treatment/stimulation, and (iii) a serum estradiol level of greater than or equal to 1.45 pmol/L prior to treatment/stimulation.

18. The method of any one claims 16-17, wherein the woman is identified as being diagnosed with oligoovulation.

19. The method of any one claims 16-18, wherein the HP-hMG is administered at a dose of 75 to 450 IU hMG per day.

20. The method of any one claims 16-18, wherein the HP-hMG is administered at a dose of 150 IU hMG per day from day 1 to at least day 5 of treatment.

21. The method any one of claims 16-20, wherein the method is effective to increase ongoing pregnancy rate after in vitro fertilization compared to controlled ovarian stimulation by administration of recombinant follicle-stimulating hormone (rFSH).

22. The method of any one of claims 16-21, further comprising administering a gonadotropin-releasing hormone antagonist (GnRH antagonist) starting on day 6 of treatment/stimulation.

23. The method of any one claims 16-22, further comprising triggering final follicular maturation by administering human chorionic gonadotropin (hCG) or a gonadotropin-releasing hormone agonist (GnRH agonist), optionally supplemented with hCG.

24. The method of any one claims 16-23, further comprising:
(a) retrieving oocyte(s), fertilizing the oocyte(s), allowing the fertilized oocyte(s) to develop to the blastocyst stage, optionally assessing the quality/morphology of the blastocyst(s), and implanting a fresh blastocyst (optionally selected based on, e.g., visual assessment of quality/morphology) in a uterus; or
(b) retrieving oocyte(s), fertilizing the oocyte(s), allowing the fertilized oocyte(s) to develop to the blastocyst stage, optionally assessing chromosomal quality of the blastocyst(s), freezing one or more or all blastocyst(s), and implanting a thawed-frozen blastocyst (e.g., a euploid blastocyst selected based on chromosomal assessment) in a uterus; or
(c) retrieving oocyte(s), freezing unfertilized oocytes(s), subsequently thawing one or more oocyte(s), fertilizing one or more or all thawed oocyte(s), allowing fertilized oocyte(s) to develop to the blastocyst stage, optionally assessing the quality/morphology of the blastocyst(s), and implanting a blastocyst (optionally selected based on, e.g., visual assessment of quality/morphology) in a uterus; or
(d) retrieving oocyte(s), freezing unfertilized oocytes(s), subsequently thawing one or more frozen oocytes, fertilizing one or more or all thawed oocyte(s), allowing fertilized oocyte(s) to develop to the blastocyst stage, optionally assessing chromosomal quality of the blastocyst(s), freezing one or more or all blastocyst(s), and implanting a thawed-frozen blastocyst (e.g., a euploid blastocyst selected based on chromosomal assessment) in a uterus.

25. The method of any one claims 16-24, wherein the woman is not anovulatory, is 21-35 years old, and has a BMI of 18-30 kg/m² at the start of treatment.

26. Use of HP-hMG in the manufacture of a medicament for the treatment of infertility in a woman identified as being diagnosed with oligoovulation and/or PCOS who has a serum AMH level≥35.7±0.5 pmol/L (≥5.0±0.2 ng/ml) prior to treatment, wherein the treatment comprises administering to the identified woman an amount of highly purified menotropin (HP-hMG) effective to stimulate follicle development.

27. The use of claim 26, wherein the woman is identified prior to treatment as having a serum estradiol level of ≥145 pmol/L (e.g., a serum estradiol level of ≥150 pmol/L) prior to treatment/stimulation, and optionally also as having one or both of a serum testosterone level of ≥1.10 nmol/L (e.g., a serum testosterone level of ≥1.14 nmol/L) prior to treatment/stimulation, and a serum luteinizing hormone (LH) level of ≥7 U/L (e.g., a serum luteinizing hormone of ≥7.55 U/L) prior to treatment/stimulation.
